(19) United States
(12) Patent Application Publication
(10) Pub. No.: US 2021/0059377 A1
(43) Pub. Date: Mar. 4, 2021

(54) Shave-preparation face and neck mask and related methods

(71) Applicant: Adam E. Tulit, Hawthorne, CA (US)

(72) Inventor: Adam E. Tulit, Hawthorne, CA (US)

(21) Appl. No.: 16/551,476

(22) Filed: Aug. 26, 2019

(51) Int. Cl. A45D 27/42; A45D 27/00

(52) U.S. Cl. CPC A45D 27/42; A45D 27/003

(57) ABSTRACT

Generally disclosed is a mask for applying heated water, ointments, or other medications to the face and neck of a user. Although the preferred usage is application of hot water for shave-preparation etc, the disclosed mask can be also used with cold water for closing the pores and cooling the face after shaving or other kind of treatments.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the generalized field of masks for applying heated water, ointments, or other medications to the face and neck of a user. The disclosed subject matter is in the preferred field of self-standing facemasks that apply hot water or other shave-preparation ointments to the face of a user to prepare sensitive facial and neck skin for the removal of facial and neck hair by a razor.

### Background of the Invention

Face shaving involves the ostensible removal of facial hair by a razor or other bladed implement. Technically, the facial hair is not removed but rather trimmed to skin level. To achieve a skin level cut of the facial hair, the sharp edge of the razor or bladed implement is often dragged along the skin surface.

Dragging a blade along the skin surface can abrade the skin and result in irritation. If the hair is bristled, then shaving can require multiple passes of the blade. So, to make the skin less susceptible to abrasion and to soften the hair so that it can be cut in a single pass, the face skin can be made supple and pores may be opened for less-bristly hair by applying heated water or steam to the face for a period of time before the act of shaving. Steaming requires a boiler or steamer whereas hot-water can usually be drawn from a bathroom sink or other water tap. Therefore, applying steam to the face is typically less convenient than the application of hot water.

Applying hot water straight from a tap or skink to the face is not always satisfactory for making the skin supple prior to shaving. Splashing or pouring water on the face does not usually expose the skin to hot water for a long enough period to make the skin supple. As a result, many have simply soaked a towel or rag with hot water and either wrapped the face with the towel or otherwise held the towel against the face.

Wrapping a towel around a face can leak water down the neck and torso of a user. Also, holding a towel against one's face is not hands-free. So, a need exists for a hands-free apparatus capable of applying hot water to the face of a user for an extended period of time with minimal leaking of hot water down the neck and torso of the user.

This need is not limited to shaving preparation. Applying medication to the face can also result in medication dripping down the neck and torso of the user. Many procedures for applying medication to the face are also not hands-free. So, a need similarly exists for a hands-free apparatus capable of applying medications or ointments to the face of a user for an extended period of time with minimal leaking of the medication or ointment down the neck and torso of the user.

### SUMMARY OF THE INVENTION

In view of the foregoing, an object of this specification is to disclose a waterproof face mask for applying hot water, medications, or other ointments to the face and neck of a user. In one embodiment, the face mask comprises: (a) a waterproof outer panel with a muzzle, neck cover, head band with ear holes, and scarf; (b) a water-retentive inner panel defined by a muzzle liner and neck flap; (c) waterproof seam tape along at least one seam of the outer panel; and (c) a seal-strip along the periphery of the neck cover that creates a water-proof seal between the neck cover and neck of a user when worn. In one mode of use: 1. the user pours hot water into muzzle, wherein the muzzle is cup-shaped; 2. the hot water is absorbed by the muzzle liner and neck flap, which may be made of a terry fabric or sponge; 3. the user puts the muzzle and neck cover over his or her face and neck so that the muzzle liner is abutted to the face of the user; 4. the headband (which may be defined by two straps that have Velcro®/hook&loop for easy attach/detach and adjustable length) is fastened at the back of his or her head so that the muzzle liner is applied to upper lip, cheeks, and chin of the user; 5. the scarf (which may be defined by two straps that have Velcro®/hook&loop for easy attach/detach and adjustable length) is fastened at the back of his or her neck so that the neck flap is confined against the neck, neck cover, and seal to retain water within the mask; 6. the mask is left in place for a time period where the mask is self-standing on his face/head including the hot water inside and the hot water steam/soften the hair and open the pores on the face for an easier and deeper shaving; 7. the waterproof outer panel retains the hot water and heat inside the muzzle and seal; 8. the waterproof seam tape along the seam keeps the water not leaking out through the seam; 9. The seal-strip interfaces with the neck skin in order to stop the water coming out between the skin of neck and the end of the mask due to the gravity; and, 10. The mask is removed after about 3-5 minutes of wearing the mask, which allows for easier shaving.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is a front-plan view of a preferred embodiment of a facemask 1000;

FIG. 2 is a bottom-plan view of the facemask 1000;

FIG. 3 is a right-plan view of the facemask 1000;

FIG. 4 is a rear-plan view of the facemask 1000;

FIG. 5 is a top-plan view of the facemask 1000;

FIG. 6 is a left-plan view of the facemask;

FIG. 7 is an illustrative view of the facemask 1000 that shows an unfurled outer panel 1100 and upturned inner panel 1200;

FIG. 7A is an exploded view of the outer panel 1100 and upturned inner panel 1200;

FIG. 8 is an illustrative view of the facemask 1000 that shows waterproof seam tape 1121;

FIG. 9 is an illustrative front view of the facemask 1000;

FIG. 10 is an illustrative rear view of the facemask 1000 that shows an intact headband 1130 and an intact scarf 1140;

FIG. 11 is an environmental front view of a second embodiment of the facemask 1000;

FIG. 12 is an environmental prospective view of the second embodiment of the facemask 1000;

FIG. 13 is an environmental side view of the second embodiment of the facemask 1000; and,

FIG. 14 is a rear view of the second embodiment of the facemask 1000.

In the figures, the following components are indicated by the corresponding reference numerals:
1000 facemask;
1100 waterproof outer panel;
1110 muzzle;
1120 neck cover;
1121 waterproof seam tape;
1130 headband;
1131 ear hole;
1140 scarf;
1150 seal-strip;
1200 water retentive inner panel
1210 muzzle liner;
1220 neck flap; and,
2000 user.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFERRED EMBODIMENTS

Generally disclosed is a mask for applying heated water, ointments, or other medications to the face of a user. The preferred embodiment of this disclosure involves a facemask for applying hot water and other ointments to the face of a user to prepare sensitive facial skin for the removal of facial hair by razor. The more specific aspects of this preferred embodiment are described with reference to the figures.

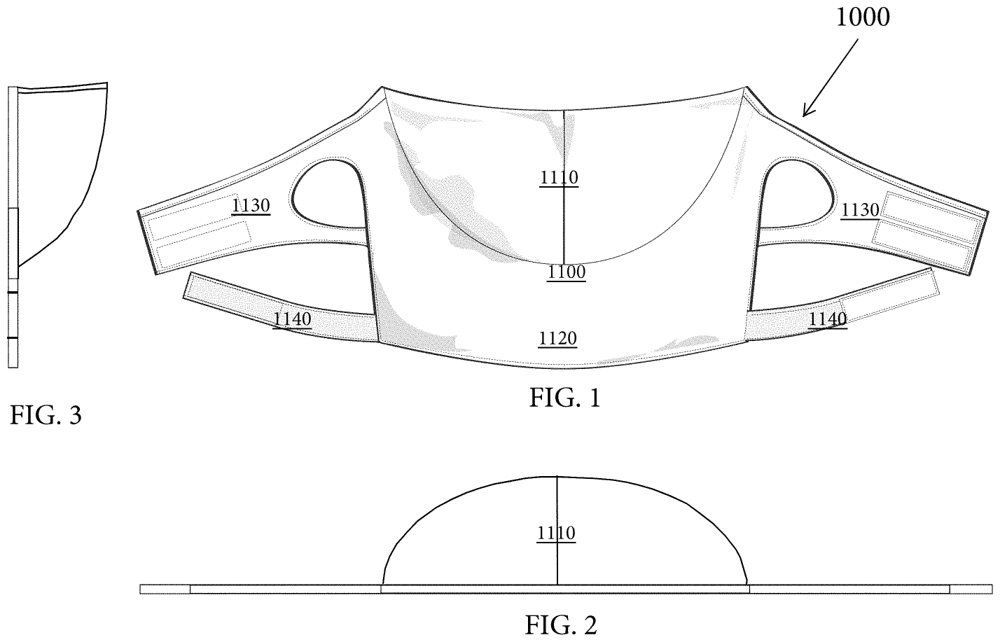

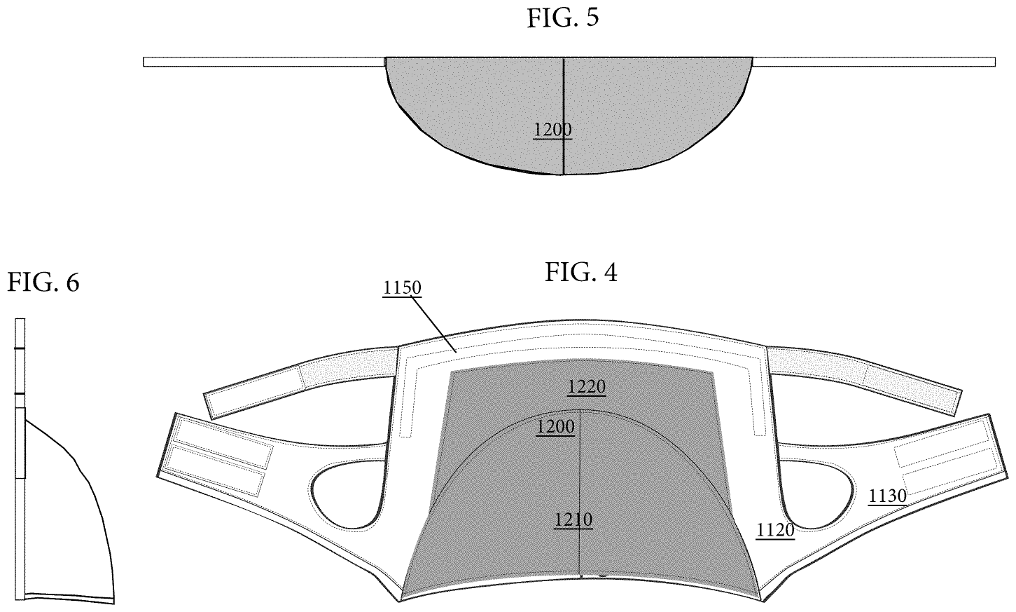

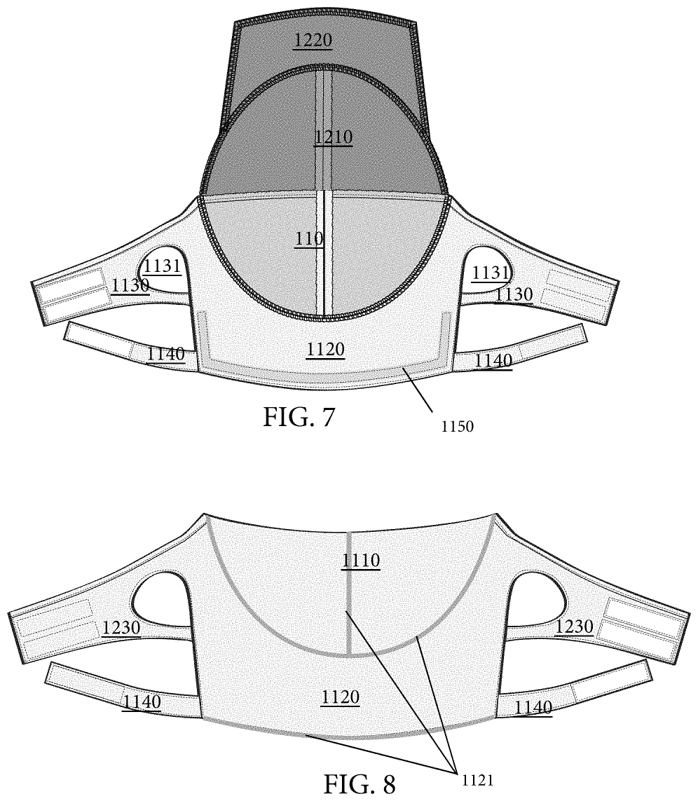

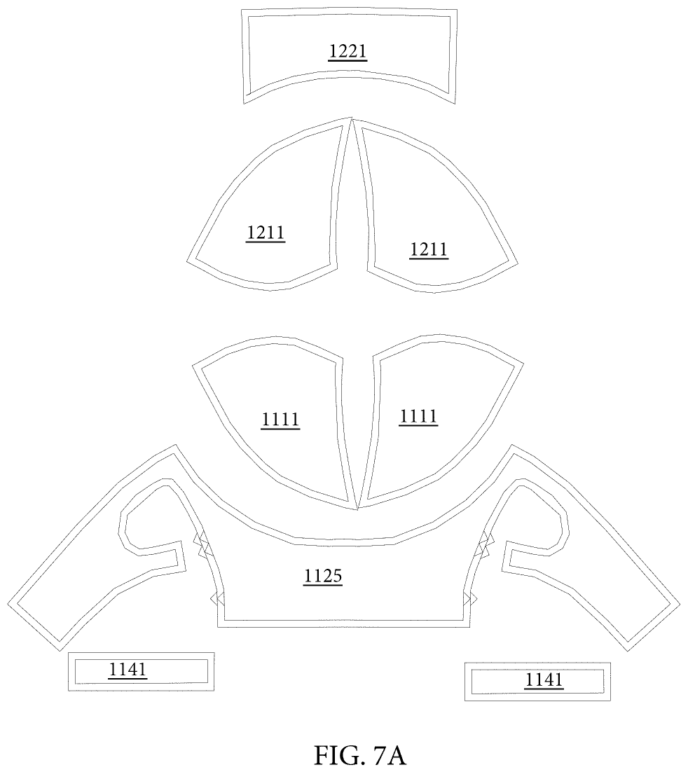

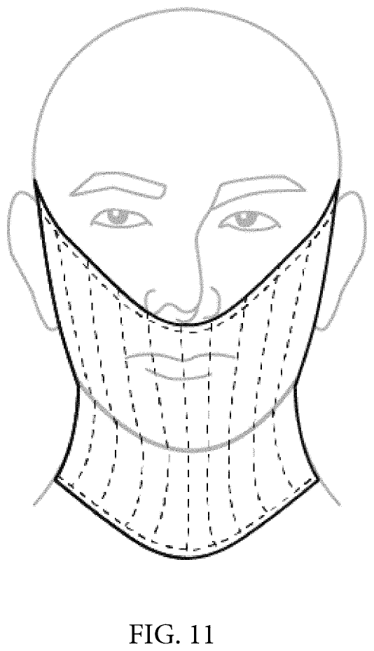

FIG. 1 is a front-plan view of a preferred embodiment of a facemask 1000. FIG. 2 is a bottom-plan view of the facemask 1000. FIG. 3 is a right-plan view of the facemask 1000. FIG. 4 is a rear-plan view of the facemask 1000. FIG. 5 is a top-plan view of the facemask 1000. FIG. 6 is a left-plan view of the facemask. FIG. 7 is an illustrative view of the facemask 1000 that shows an unfurled outer panel 1100 and upturned inner panel 1200. FIG. 7A is an exploded view of the outer panel 1100 and upturned inner panel 1200. FIG. 8 is an illustrative view of the facemask 1000 that shows waterproof seam tape 1121. As shown in FIGS. 1 through 8, the facemask 1000 comprises (a) a waterproof outer panel 1100 with a muzzle 1110, neck cover 1120, head band 1130 with ear hole 1131, and scarf 1140; (b) a water-retentive inner panel 1200 defined by a muzzle liner 1210 and neck flap 1220; (c) waterproof seam tape 1121 along at least one seam 1101 of the outer panel 1100; and (c) a seal-strip 1150 along the periphery of the neck cover 1130 that creates a water-proof seal between the neck cover and neck of a user (not shown) when worn. See, e.g., FIGS. 11 through 14.

Referring to FIGS. 1 through 8, the water proof outer panel 1100 has an outside (see FIGS. 1 through 3, 6, and 8) and an inside (see FIGS. 4, 5, and 7). FIG. 7A shows that the outer panel 1100 is defined by two muzzle cutouts 1111, a neck cover and headband cutout 1125, and two scarf cutouts 1141. As shown in FIG. 8, the cutouts 1111, 1125, 1141 can be sewn together to form the outer panel 1000 wherein the seams of the assembly are provided with waterproof seam tape 1121 to make the seams substantially water tight. Suitably, hooks from a hook and loop fastener may be placed on one side of the outer panel 1100 at the end of one of the headband strips 1130 and loops of the hook and loop fastener may be placed on the other end of the headband strips 1130. Suitably, hooks from a hook and loop fastener may be placed on one side of the outer panel 1100 at the end of one of the scarf strips 1140 and loops of the hook and loop fastener may be placed on the other end of the scarf strips 1140. As shown in FIGS. 4 and 7, a seal-strip 1150 (of e.g., silicone) may be provided around the periphery of the neck cover 1200 on the inside of the outer panel 1100. Although the outer panel 1100 is shown as sewn together cutouts, the outer panel may be formed of one piece in an alternate embodiment.

Suitably, the outer panel 1100 may be made of water proof fabric or fabric that has been treated so that it is waterproof or otherwise resistant to penetration by water. These fabrics may be natural or synthetic fabrics that are laminated or coated with materials such as Polyurethane, rubber, silicone elastomer, flouromers, wax, rubber or polyvinyl chloride. Waterproofing could also be accomplished via application to the outer panel 1100 of durable water repellants.

Still referring to FIGS. 1 through 8, the water retentive inner panel 1200 has a first side (see FIGS. 4 and 5) and a flipside (see FIG. 7). FIG. 7A shows that the inner panel is defined by two muzzle liner cutouts 1211 and a neck flap cutout 1221. As shown in FIG. 8, the cutouts 1111, 1125, 1141 can be sewn together to form the inner panel 1200. As shown in FIG. 7, the inner panel may be sewn to the outer panel along an upper periphery of the muzzle 1120 with, e.g., a clean finish top edge. Although the inner panel 1200 is shown as sewn together cutouts, the inner panel may be formed of one piece in an alternate embodiment.

Suitably, the inner panel 1200 may be made of water retentive fabric or sponges. These fabrics may be natural or synthetic fabrics that retain water. Materials common to absorbent bath towels or dish rags and sponges will be well known to those of skill in the art. Waterproofing could also be accomplished via application to the outer panel 1100 of durable water repellants.

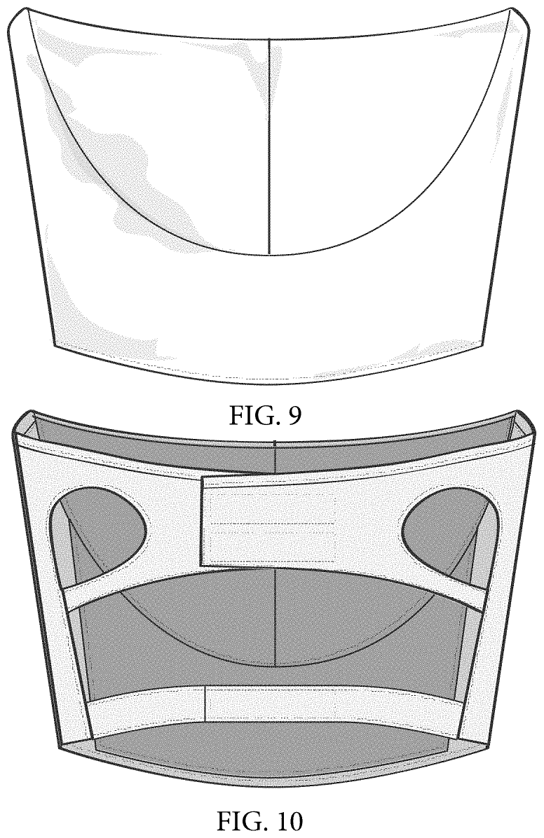

FIGS. 9 and 10 are illustrative front and rear view of the facemask 1000. As shown, the hook and loop portions of the hook and loop fastener may be drawn together to form the headband 1130 and scar 1140. Suitably, the headband 1130 and scarf 1140 may be adjustable to the size of the user's head and neck.

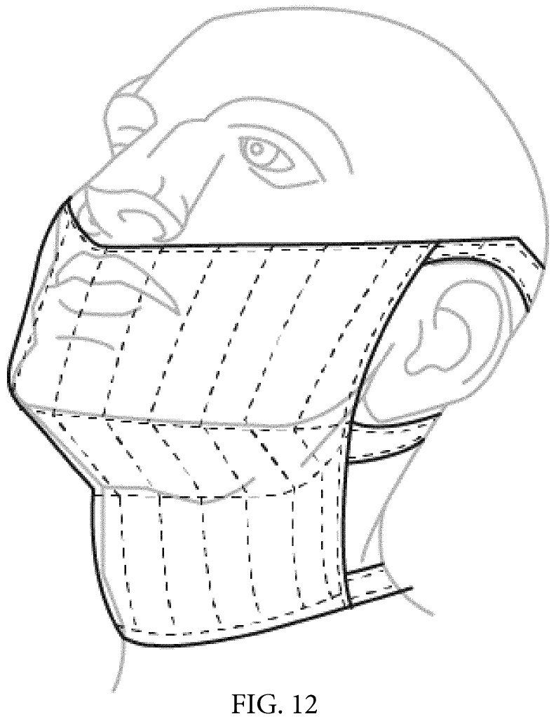

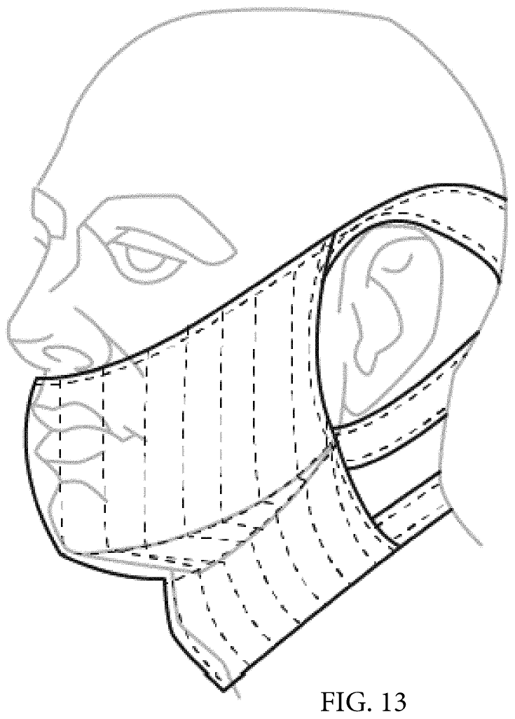

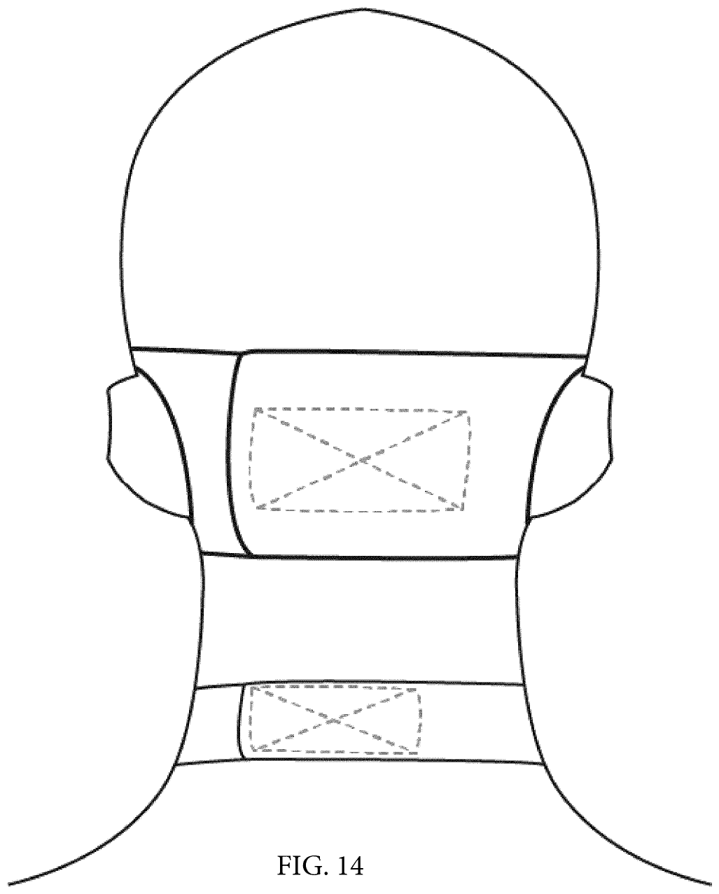

FIG. 11 is an environmental front view of a second embodiment of the facemask 1000. FIG. 12 is an environmental prospective view of the second embodiment of the facemask 1000. FIG. 13 is an environmental side view of the second embodiment of the facemask 1000. FIG. 14 is a rear view of the second embodiment of the facemask 1000.

FIGS. 11 through 14 show a preferred mode of use of the facemask 1000: 1. the user pours hot water into muzzle wherein the muzzle is cup-shaped; 2. the hot water is absorbed by the muzzle liner and neck flap, which may be made of a terry fabric or sponge; 3. the user puts the muzzle and neck cover over his or her face and neck so that the muzzle liner abutted to the face of the user; 4. the headband (which may be defined by two straps that have Velcro®/hook&loop for easy attach/detach and adjustable length) is fastened at the back of his or her head so that the muzzle liner is applied to upper lip, cheeks, and chin of the user; 5. the scarf (which may be defined by two straps that have Velcro®/hook&loop for easy attach/detach and adjustable length) is fastened at the back of his or her neck so that the neck flap is confined against the neck, neck cover, and seal to retain water within the mask; 6. the mask is left in place for a period of time where the mask is self-standing on his face/head including the hot water inside and the hot water steam/soften the hair and open the pores on the face for an easier and deeper shaving; 7. the waterproof outer panel retains the hot water and heat inside the muzzle and seal; 8. the waterproof seam tape along the seam keeps the water not leaking out through the seam; 9. The seal-strip 1150 interfaces with the neck skin in order to stop the water coming out between the skin of neck and the end of the mask due to the gravity; and, 10. The mask is removed after about 3-5 minutes of wearing the mask and easier shaving is accomplished.

FIGS. 11 through 14 show the face and neck mask is designed to cover the upper and lower lips, the left and right cheeks, the chin, and the neck of a user without covering the user's nose. The embodiment of the mask depicted in FIGS. 1 through 10 is also designed to cover the upper and lower lips, the left and right cheeks, the chin, and the neck of a user without covering the user's nose. The face and neck masks have a headband with ear holes and a neck strap or scarf.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments. In particular, it should be mentioned that although the preferred usage is application of hot water for shave-preparation etc, the disclosed mask can be also used with cold water for closing the pores and cooling the face after shaving or other kind of treatments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A face mask comprising:
(a) a waterproof outer panel with a muzzle, neck cover, head band with ear holes, and scarf;
(b) a water-retentive inner panel defined by a muzzle liner and neck flap;
(c) waterproof seam tape along at least one seam of the outer panel; and
(c) a seal-strip along the periphery of the neck cover that creates a water-proof seal between the neck cover and neck of a user when worn.

2. A method of treating the skin comprising the steps of:
1. pouring hot water into a muzzle of a facemask wherein the muzzle is cup-shaped and the facemask is further comprised of a neck cover with a seal-strip, a headband, and a scarf;
2. absorbing hot water by a muzzle liner disposed in the muzzle and a neck flap that is adjacent the neck cover;
3. putting the muzzle and neck cover over a human face and neck so that the muzzle liner abuts the human face;
4. securing the facemask around a head associated with the human face via the headband so that the muzzle liner is applied to an upper lip, cheeks, and a chin of the human face;
5. securing the scarf around a neck associated with the human neck so that the neck flap is confined against the neck, neck cover, and seal-strip to retain water within the mask;
6. leaving the mask in place for a time period wherein the mask is self-standing on his face/head includes the hot water to soften the hair and open the pores on the human face for an easier and deeper shaving;
7. further using a waterproof outer panel to retain the hot water and heat inside the muzzle and seal strip; and,
10. removing the facemask from the human face after about 3-5 minutes.
